(19) United States
(12) Patent Application Publication
(10) Pub. No.: US 2011/0184058 A1
(43) Pub. Date: Jul. 28, 2011

(54) SYNTHESIS OF 6,7-DIHYDRO-1H-INDENO[5, 4-B] FURAN-8(2H)-ONE AS INTERMEDIATE IN THE PREPARATION OF REMELTEON

(75) Inventor: Jerome Cluzeau, Ljubljana (SI)

(73) Assignee: Lek Pharmaceuticals D.D., Ljubljana (SI)

(21) Appl. No.: 13/054,025

(22) PCT Filed: Jul. 13, 2009
(86) PCT No.: PCT/EP09/58920

§ 371 Date: Mar. 28, 2011

(30) Foreign Application Priority Data
Jul. 14, 2008 (EP) 08160341.7

(51) Int. Cl. A61K 31/343; C07D 307/78; A61P 25/00

(52) U.S. Cl. 514/468; 549/458; 549/471

(57) ABSTRACT

The present invention describes the preparation of 6,7-dihydro-1H-indeno[5,4-b]furan-8(2H)-one, a key intermediate in preparation of ramelteon. The present invention also describes further preceding intermediate compounds useful for the synthesis of 6,7-dihydro-1H-indeno[5,4-b]furan-8(2H)-one.

### FIELD OF THE INVENTION

The present invention relates in general to the field of organic chemistry and in particular to the preparation of 6,7-dihydro-1H-indeno[5,4-b]furan-8(2H)-one, a key intermediate in preparation of ramelteon.

### BACKGROUND OF THE INVENTION

Ramelteon, (S)-N-[2-(1,6,7,8-tetrahydro-2H-indeno-[5,4-b]furan-8-yl)ethyl]propionamide, is a melatonin receptor agonist with both high affinity for melatonin MT1 and MT2 receptors and selectivity over the MT3 receptor. Ramelteon demonstrates full agonist activity in vitro in cells expressing human MT1 or MT2 receptors, and high selectivity for human MT1 and MT2 receptors compared to the MT3 receptor. The activity of ramelteon at the MT1 and MT2 receptors is believed to contribute to its sleep-promoting properties, as these receptors, acted upon by endogenous melatonin, are thought to be involved in the maintenance of the circadian rhythm underlying the normal sleep-wake cycle.

The synthesis of ramelteon is disclosed in EP885210B1, EP1792899A1 and J. Med. Chem. 2002, 45, 4222-4239. Ramelteon is synthesized in two parts; first the synthesis of the tricyclic core with the key intermediate 6,7-dihydro-1H-indeno[5,4-b]furan-8(2H)-one and then the side chain with the introduction of the chirality and amide function. The synthesis of 6,7-dihydro-1H-indeno[5,4-b]furan-8(2H)-one intermediate consists of 6 or 7 steps using 2,3-benzofuran as starting material and in several steps involves the use of small to large excess of halogenated reagents, which are toxic and environmentally unfriendly.

There is a need for efficient synthesis of 6,7-dihydro-1H-indeno[5,4-b]furan-8(2H)-one, a key intermediate in preparation of ramelteon.

### DISCLOSURE OF THE INVENTION

One aspect of present invention is a process for preparing the compound of formula V

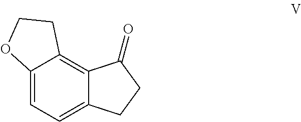

from a compound of formula III

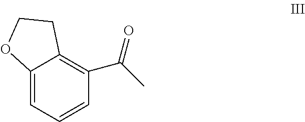

comprising a step of converting the ethanone group of the compound of formula III into acrylate group, and a subsequent step of cyclization of the acrylate group-containing compound to give the compound of formula V.

In another aspect of this invention said process for preparing the compound of formula V comprises the steps of
a.) reacting a compound of formula III

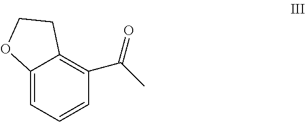

with paraformaldehyde in the presence of ammonium salt
in organic solvent and
b.) contacting the solution with strong inorganic acid.

In another aspect of this invention said ammonium salt is
R¹R²NH₂⁺X⁻, wherein R¹ and R² are each independently selected from substituted and unsubstituted alkyl, cycloalkyl, aryl, arylalkyl and arylcycloalkyl; and X is halogen or R³CO₂, wherein R³ is substituted or unsubstituted alkyl or aryl, wherein substitution is preferably halogen, more preferably polyhalogen substitution.

As used herein, the terms alkyl, cycloalkyl, aryl, arylalkyl or arylcycloalkyl may denote organic groups contain 1 to 12, preferably 1 to 8 and more preferably 1 to 6 carbon atoms. A preferred residues R₁ and R₂ are lower alkyl with 1 to 6 carbon atoms such as methyl, ethyl, propyl, isopropyl, n-butyl, tert-butyl, pentyl, hexyl or phenyl. Substitution may include halogen such as fluoro, chloro, bromo or the like, without being limited thereto.

In another aspect of this invention the said acrylate group-containing compound is a compound of formula IV

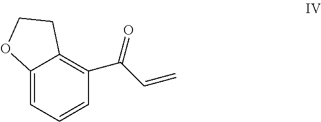

In another aspect of this invention the compound of formula IV is obtained in solution, preferably in an apolar solvent, suitably selected from alkanes, ethers and chlorinated solvents.

In another aspect of this invention said compound of formula III is prepared by a process comprising
reacting a compound of formula II

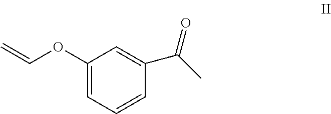

with primary amine and then further reacting it with metal catalyst

In another aspect of this invention said compound of formula II is prepared by a process comprising OH protection of a compound of formula I

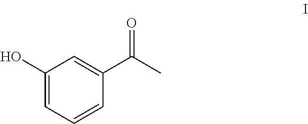

with vinyl group, preferably by reaction with vinyl acetate.

Another aspect of this invention is a compound of formula III.

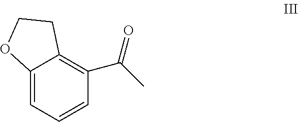

Another aspect of this invention is a compound of formula IV.

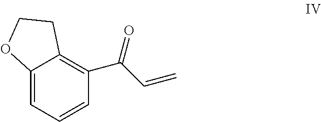

Another aspect of this invention is use of any compound selected from 1-(3-hydroxyphenyl)ethanone (compound I), 1-(3-(vinyloxy)phenyl)ethanone (compound II), 1-(2,3-dihydrobenzofuran-4-yl)ethanone (compound III), and 1-(2,3-dihydrobenzofuran-4-yl)prop-2-en-1-one (compound IV) for the preparation of 6,7-dihydro-1H-indeno[5,4-b]furan-8(2H)-one (V) and/or ramelteon.

Another aspect of this invention is a process for preparing the compound of formula V comprising the steps of:

a.) preparing a compound of formula III by reacting a compound of formula II with primary amine
b.) reacting a compound of formula III with paraformaldehyde in the presence of ammonium salt, R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent
c.) contacting the solution with strong inorganic acid:

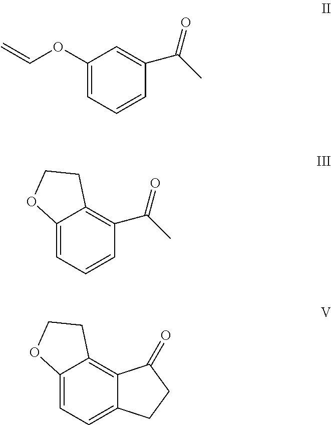

Another aspect of this invention is a process for preparing the compound of formula V comprising the steps of:

a.) preparing a compound of formula II by reacting compound of formula I with vinyl acetate
b.) preparing a compound of formula III by reacting a compound of formula II with primary amine
c.) reacting a compound of formula III with paraformaldehyde in the presence of ammonium salt, R¹R²NH₂+X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent
d.) contacting the solution with strong inorganic acid

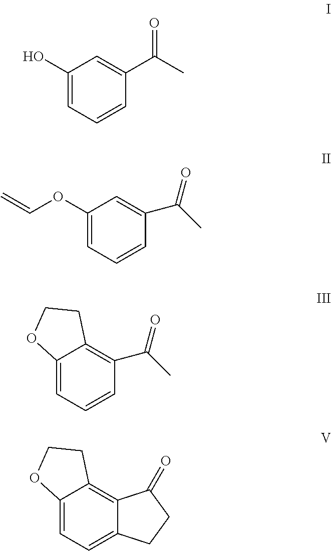

Another aspect of this invention is a process for preparing the compound of formula V comprising the steps of:

a.) preparing a compound of formula II by reacting compound of formula I with vinyl acetate
b.) preparing a compound of formula III by reacting a compound of formula II with primary amine.
c.) reacting a compound of formula III with paraformaldehyde in the presence of ammonium salt, R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent
d.) obtaining the reaction product of c) comprising a compound of formula IV in solution of said organic solvent
e.) contacting the solution with strong inorganic acid

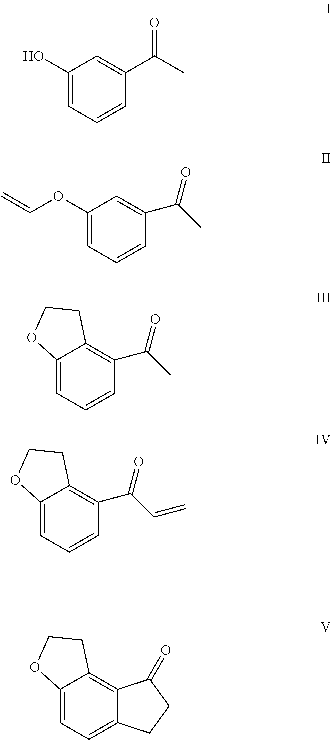

Another aspect of this invention is a process for the preparation of ramelteon, comprising the steps of:

carrying out a process for preparing the compound of formula V according to any one of aspects of this invention
subjecting the compound of formula V to further synthesis steps to yield ramelteon.

Another aspect of this invention is a process for the preparation of a pharmaceutical composition comprising ramelteon as active ingredient, comprising the steps of: preparing ramelteon according to the process according to previous aspect and admixing the thus prepared ramelteon with at least one pharmaceutically acceptable excipient.

The invention solves the problem of long and tedious synthesis of tricycle 6,7-dihydro-1H-indeno[5,4-b]furan-8(2H)-one intermediate. A process according to this invention is short and efficient with yields that are industrially applicable and competitive. It uses cheap starting materials and involves only four steps. Compared to prior art processes reduced amounts of halogenated reagents are used.

### DETAILED DESCRIPTION OF THE INVENTION

The present invention is described in more detail while referring to preferred embodiments and examples, which are presented however for illustrative purposes and shall not be construed to limit the invention in any way.

Reaction Scheme 1 illustrates a preferred embodiment of the process according to the present invention for preparing 6,7-dihydro-1H-indeno[5,4-b]furan-8(2H)-one intermediate of formula V, a key intermediate in preparation of ramelteon.

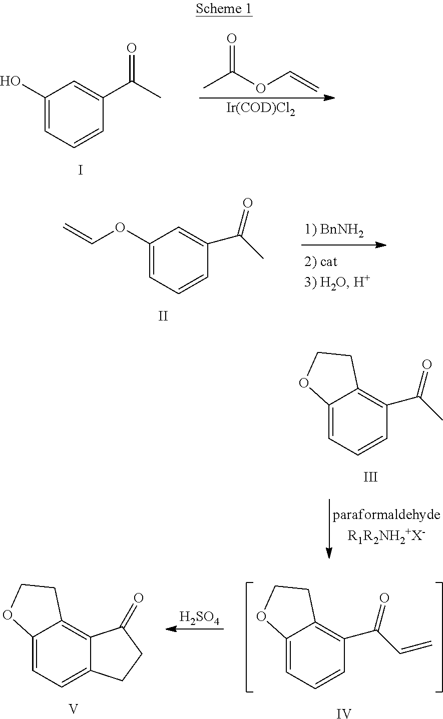

According to the preferred embodiment of Scheme 1 compound of formula II is prepared by protecting a compound of formula I with vinyl group. Preferably vinyl acetate in the presence of Ir(COD)Cl)₂ is used. The reaction is preferably performed at about 50° C. to 120° C. for 2 to 4 hours.

Further according to the preferred embodiment of Scheme 1, compound of formula III is prepared by reacting a compound of formula II with primary amine, preferably benzylamine. The reaction is preferably performed in the presence of a catalyst, preferably selected from the group consisting of metal catalyst, such as for example rhodium or ruthenium, or from derivative of said metal, such as for example Cp* or phosphines.

The reaction is preferably performed at about 50° C. to 200° C. for, more preferably at about 100° C. to 180° C., most preferably at about 140° C. to 160° C.

Further according to the preferred embodiment of Scheme 1, a compound of formula III is reacted with paraformaldehyde in the presence of an ammonium salt of formula R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl), such as for example TADCA (dicyclohexylammonium 2,2,2-trifluoroacetate) or TAMA (N-methylanilinium 2,2,2-trifluoroacetate).

The excess of the ammonium salt (up to 1 equivalent) can be used.

The reaction is preferably performed in aprotic solvent for 1 to 36 hours, more preferably for 4-12 hours, at about 60 to 120° C.

At this stage acrylate intermediate IV can be effectively obtained in the form of a solution in organic solvent. The organic solvent is suitably an apolar solvent and is preferably selected from the group of alkanes, ethers or chlorinated solvents. Advantageously, it is not necessary that intermediate IV is isolated.

The solution is then reacted with strong inorganic acid, preferably sulfuric acid, at a temperature between 0 to 100° C., preferably 30° C. to 70° C. to give a compound of formula V.

The key intermediate compound of formula V, 6,7-dihydro-1H-indeno[5,4-b]furan-8(2H)-one (V), can then be subjected to further synthesis steps to yield ramelteon by synthesis route known to or readily devisable by a person skilled in the art, suitably involving the introduction of the side chain having chirality and amide function. The documents mentioned infra are incorporated herein by way of reference. For example, the following synthesis route may be applied:

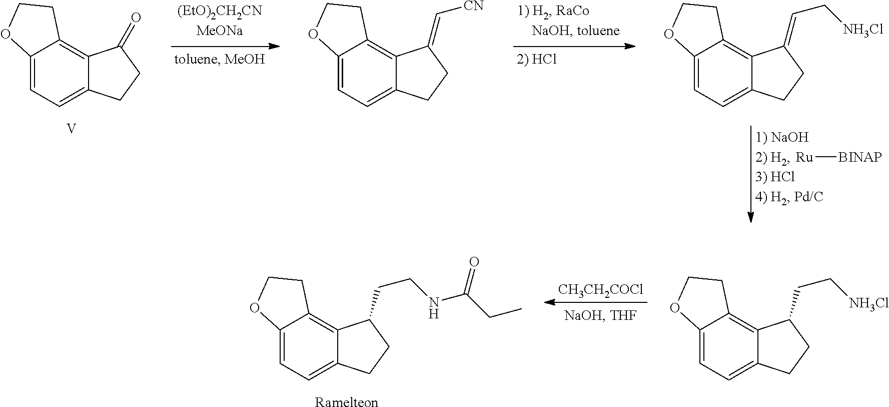

For preparing a pharmaceutical composition comprising ramelteon as active ingredient, first ramelteon is provided by the process as described above, and then the thus prepared ramelteon is admixed with at least one suitable pharmaceutically acceptable excipient. Pharmaceutically acceptable excipients may be selected from the group consisting of binders, diluents, disintegrating agents, stabilizing agents, preservatives, lubricants, fragrances, flavoring agents, sweeteners and other excipients known in the field of the pharmaceutical technology. Preferably, carriers and excipients may be selected from the group consisting of lactose, microcrystalline cellulose, cellulose derivatives, e.g. hydroxypropylcellulose, polyacrylates, calcium carbonate, starch, colloidal silicone dioxide, sodium starch glycolate, talc, magnesium stearate, polyvinylpyrrolidone, polyethylene glycol and other excipients known in the field of the pharmaceutical technology.

### Experimental Procedures

### Example 1

### Preparation of 1-(3-(vinyloxy)phenyl)ethanone (II)

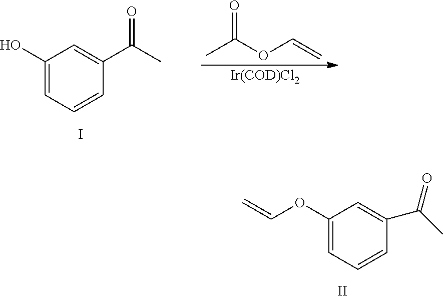

1-(3-hydroxyphenyl)ethanone (I) (5 g, 36.8 mmol) was suspended in dry toluene (37 mL), dry sodium carbonate (2.34 g, 0.6 eq) and (Ir(COD)Cl)₂ (247 mg, 0.01 eq) were added. Vinyl acetate (6.8 mL, 2 eq) was finally added and the reaction was heated at 100° C. for 2 h. Reaction was cooled down to room temperature, filtered and concentrated. Residue was purified by flash chromatography (100% hexane to 95/5 hexane/EtOAc) to give 1-(3-(vinyloxy)phenyl)ethanone (5.05 g, 85%). ¹H NMR δ (CDCl₃) 7.65 (d, 1H, J=7.7 Hz), 7.56 (t, 1H, J=2.0 Hz), 7.40 (t, 1H, J=8.0 Hz), 7.19 (dd, 1H, J=2.5 Hz, J=8.1 Hz), 6.66 (dd, 1H, J=6.0 Hz, J=13.7 Hz), 4.80 (dd, 1H, J=1.8 Hz, J=13.7 Hz), 4.50 (dd, 1H, J=1.8 Hz, J=6.0 Hz), 2.58 (s, 3H). ¹³C NMR δ (CDCl₃) 197.3, 156.9, 147.5, 138.6, 129.8, 123.1, 121.8, 116.0, 96.1, 26.6.

Large Scale Preparation of 1-(3-(vinyloxy)phenyl)ethanone (II)

1-(3-hydroxyphenyl)ethanone (I) (204 g, 1.5 mole) was suspended in dry toluene (1.5 L), dry sodium carbonate (95.4 g, 0.6 eq) and (Ir(COD)Cl)₂ (10 g, 0.01 eq) were added. Vinyl acetate (276 mL, 2 eq) was finally added and the reaction was heated at 100° C. for 2 h 30 min. Reaction was cooled down to room temperature, filtered on activated carbon. Activated carbon was rinsed with toluene and EtOAc. Organic solvents were concentrated. Residue was purified by flash chromatography (100% hexane to 87/13 hexane/EtOAc) to give 1-(3-(vinyloxy)phenyl)ethanone (173 g, 71%).

### Example 2

### Preparation of 1-(2,3-dihydrobenzofuran-4-yl)ethanone (III)

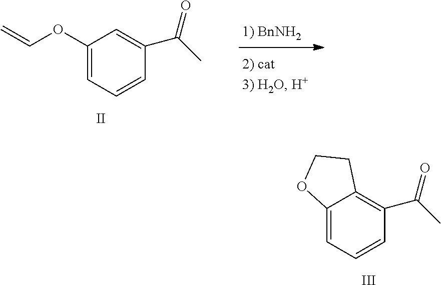

1-(3-(vinyloxy)phenyl)ethanone (II) (1.62 g, 10 mmol) was dissolved in dry toluene (100 mL), 4 Å molecular sieves (10 g, 1 g/mmol) and benzylamine (1.1 mL, 10 mmol) were added and the reaction was heated at reflux for 18 h. Reaction was cooled down to room temperature, filtered and concentrated. Residue was dissolved in toluene (100 mL), Ph₃PRhCl (462 mg, 0.05 eq) was added and reaction was heated for 24 h at 150° C. in a pressure reactor. Reaction was cooled down to room temperature, 1N HCl (100 mL) was added and the reaction was stirred for 2 h. Phases were separated and organic phase was washed successively with 1N HCl, water and brine. Organic phase was dried over MgSO₄, filtered, concentrated and purified by flash chromatography to give 1-(2,3-dihydrobenzofuran-4-yl)ethanone (1.17 g, 72%). ¹H NMR δ (CDCl₃) 7.35 (dd, 1H, J=0.8 Hz, J=7.8 Hz), 7.19 (t, 1H, J=7.9 Hz), 6.95 (d, 1H, J=8.0 Hz), 4.57 (t, 2H, J=8.8 Hz), 3.52 (t, 2H, J=8.8 Hz), 2.57 (s, 3H). ¹³NMR δ (CDCl₃) 198.8, 161.0, 133.8, 128.2, 127.9, 121.4, 113.4, 71.6, 31.0, 27.6.

Large scale preparation of 1-(2,3-dihydrobenzofuran-4-yl)ethanone (III)

1-(3-(vinyloxy)phenyl)ethanone (H) (40 g, 247 mmol) was dissolved in dry toluene (2.4 L), 4 Å molecular sieves (247 g, 1 g/mmol) and benzylamine (26.9 mL, 1 eql) were added and the reaction was heated at reflux for 18 h. Reaction was cooled down to room temperature and filtered. Ph₃PRhCl (2,328 g, 0.01 eq) was added and reaction was heated for 4 days at 140° C. in a pressure reactor. Reaction was cooled down to room temperature, 1N HCl (2.5 L) was added and the reaction was stirred for 2 h. Phases were separated and aqueous phase was re-extracted twice with toluene (1 L). Combined organic phase were washed successively with 1N HCl and water. Organic phase was dried over MgSO₄, filtered, concentrated and purified by distillation under reduced pressure to give 1-(2,3-dihydrobenzofuran-4-yl)ethanone (26.22 g, 66%).

### Example 3

### Preparation of 6,7-dihydro-1H-indeno[5,4-b]furan-8(2H)-one (V)

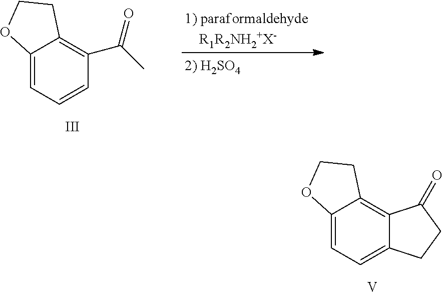

1-(2,3-dihydrobenzofuran-4-yl)ethanone (III) (1 g, 6.2 mmol) was dissolved in dioxane (9 mL). TADCA (dicyclohexylammonium 2,2,2-trifluoroacetate) (1.82 g, 1 eq) and paraformaldehyde (0.611 g, 1.1 eq) were added. The reaction was heated at 100° C. for 2 h. A second portion of TADCA (0.91 g, 0.5 eq) and paraformaldehyde (0.333 g, 0.6 eq) were added and the reaction was heated at 100° C. for 2 h. Reaction was partitioned between water (20 mL) and pentane (30 mL). Aqueous phase was re-extracted 4 times with pentane (10 mL). Combined pentane phases were washed with water and brine, dried over MgSO₄. Solution was diluted to 100 mL with pentane. This solution was added dropwise to a pre-heated solution of sulfuric acid at 67° C. (10 mL) under nitrogen stream. At the end of addition, the reaction was stirred for 30 min. Reaction was cooled down to room temperature and poured on iced water (50 mL). Solution was extracted 5 times with MTBE. Combined organic phases were washed with water, NaHCO₃ 1M and brine, dried over MgSO₄ and concentrated. Purification by flash chromatography furnished pure 6,7-dihydro-1H-indeno[5,4-b]furan-8(2H)-one. ¹H NMR δ (CDCl₃) 7.21 (dd, 1H, J=0.9 Hz, J=9.0 Hz), 7.02 (d, 1H, J=8.2 Hz), 4.66 (t, 2H, J=8.9 Hz), 3.48 (t, 2H, J=8.9 Hz), 3.08 (dd, 2H, J=4.9 Hz, J=6.0 Hz), 2.69 (m, 2H). ¹³C NMR δ (CDCl₃) 207.5, 160.2, 147.1, 133.6, 125.6, 123.9, 115.6, 72.3, 37.1, 28.4, 25.4.

Large scale preparation of 6,7-dihydro-1H-indeno[5,4-b]furan-8(2H)-one (V)

1-(2,3-dihydrobenzofuran-4-yl)ethanone (III) (10 g, 61.7 mmol) was dissolved in dioxane (50 mL). N-methylaniline (0.33 mL, 0.05 eq), TFA (0.24 mL, 0.05 eq) and paraformaldehyde (1.67 g, 0.3 eq) were added. The reaction was heated at 100° C. for 4 h. N-methylaniline (0.33 mL, 0.05 eq) and TFA (0.24 mL, 0.05 eq) were added again after the first, second and third hour Reaction was partitioned between 1:1 brine:water (200 mL) and pentane (166 mL). Aqueous phase was re-extracted 3 times with pentane (110 mL). Combined pentane phases were washed with water and brine, dried over MgSO₄. Solution was diluted to a total volume of 500 mL of pentane. This solution was added dropwise to a pre-heated solution of sulfuric acid at 67° C. (66 mL) under nitrogen stream. At the end of addition, the reaction was stirred for 30 min. Reaction was cooled down to room temperature and ice (116 mL) and MTBE (116 mL) were added. Solution was stirred overnight and extracted 3 times with 1:1 MTBE:EtOAc (150 mL). Combined organic phases were washed with water, NaHCO₃ 1M (170 mL), dried over MgSO₄ and concentrated. Purification by flash chromatography furnished 1-(2,3-dihydrobenzofuran-4-yl)ethanone (3.47 g, 35% recovered material) and pure 6,7-dihydro-1H-indeno[5,4-b]furan-8(2H)-one (2.85 g, 27%).

1. A process for preparing the compound of formula V 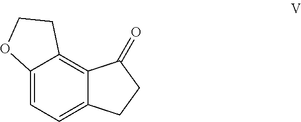
from a compound of formula III 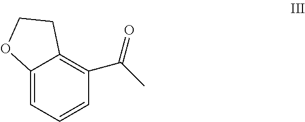
comprising a step of converting the ethanone group of the compound of formula III into acrylate group, and a subsequent step of cyclization of the acrylate group-containing compound to give the compound of formula V.

2. The process according to claim 1 comprising the steps of
a.) reacting a compound of formula III 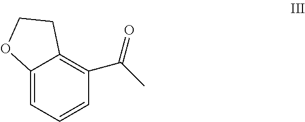
with paraformaldehyde in the presence of ammonium salt
in organic solvent and
b.) contacting the solution with strong inorganic acid

3. The process according to claim 2, wherein said ammonium salt is R¹R³NH₂⁺X⁻, wherein R¹ and R² are each independently selected from substituted or unsubstituted alkyl, cycloalkyl, aryl, arylalkyl or arylcycloalkyl; and
X is halogen or R³CO₂, wherein R³ is substituted or unsubstituted alkyl or aryl, wherein substitution is preferably halogen, more preferably polyhalogen substitution.

4. The process of claim 1, wherein the acrylate group-containing compound is a compound of formula IV

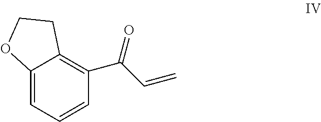

5. The process of claim 4, wherein the compound of formula IV is obtained in solution, which is an apolar solvent selected from the group consisting of alkanes, ethers and chlorinated solvents.

6. The process of claim 1, wherein said compound of formula III is prepared by a process comprising reacting a compound of formula II 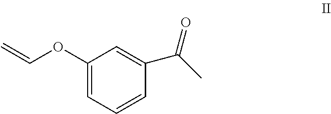
with primary amine and then further reacting it with metal catalyst

7. The process of claim 6, wherein said compound of formula II is prepared by a process comprising OH protection of a compound of formula I 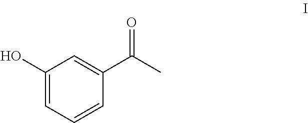
with vinyl group, preferably by reaction with vinyl acetate.

8. A compound of formula III.

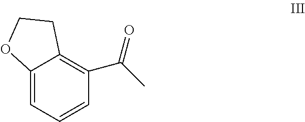

9. A compound of formula IV.

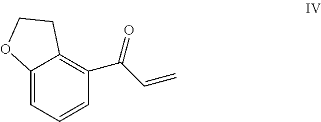

10. (canceled)

11. A process for preparing the compound of formula V comprising the steps of:
a.) preparing a compound of formula III by reacting a compound of formula II with primary amine;
b.) reacting a compound of formula III with paraformaldehyde in the presence of ammonium salt, R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent;
c.) contacting the solution with strong inorganic acid:

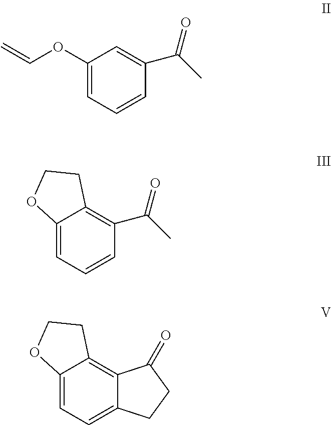

12. A process for preparing the compound of formula V comprising the steps of:
a.) preparing a compound of formula II by reacting compound of formula I with vinyl acetate;
b.) preparing a compound of formula III by reacting a compound of formula II with primary amine;
c.) reacting a compound of formula III with paraformaldehyde in the presence of ammonium salt, R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent;
d.) contacting the solution with strong inorganic acid

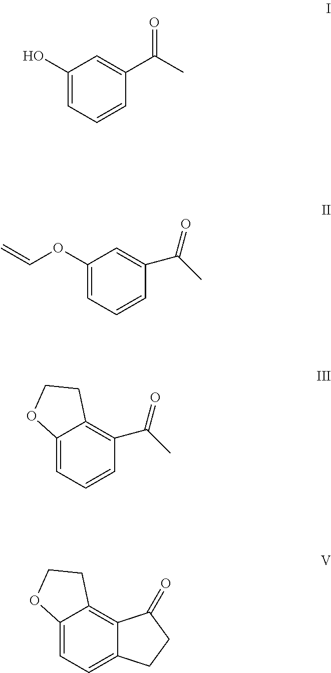

13. A process for preparing the compound of formula V comprising the steps of:
a.) preparing a compound of formula II by reacting compound of formula I with vinyl acetate;
b.) preparing a compound of formula III by reacting a compound of formula II with primary amine;
c.) reacting a compound of formula III with paraformaldehyde in the presence of ammonium salt, R¹R²NH₂⁺X⁻, (wherein R¹ and R² are each independently selected from alkyl, cycloalkyl, aryl, arylalkyl and arylcycloalkyl; and X is halogen or R³CO₂, wherein R³ is one of alkyl, aryl, polyhaloalkyl) in organic solvent;
d.) obtaining the reaction product of c) comprising a compound of formula IV in solution of said organic solvent; and
e.) contacting said solution with strong inorganic acid

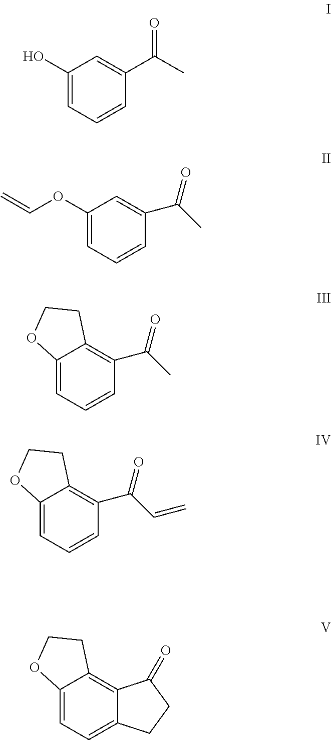

14. A process for the preparation of ramelteon, comprising the steps of:
carrying out a process for preparing the compound of formula V according to any one of claims 1-7, 11-13; and
subjecting the compound of formula V to further synthesis steps to yield ramelteon

15. A process for the preparation of a pharmaceutical composition comprising ramelteon as active ingredient, comprising the steps of:
preparing ramelteon according to the process according to claim 14, and admixing the thus prepared ramelteon with at least one pharmaceutically acceptable excipient.
